(19) United States
(12) Patent Application Publication
(10) Pub. No.: US 2023/0293343 A1
(43) Pub. Date: Sep. 21, 2023

(54) MEDICAL DEVICE SYSTEMS FOR THERMAL THERAPY

(71) Applicant: Medicool Technologies Inc., Rochester, MN (US)

(72) Inventors: Jeffrey Rynbrandt, Traverse City, MI (US); Charles Ritrivi, Houston, TX (US)

(21) Appl. No.: 18/118,490

(22) Filed: Mar. 7, 2023

Related U.S. Application Data
(60) Provisional application No. 63/320,862, filed on Mar. 17, 2022.

(51) Int. Cl. A61F 7/12; A61B 5/361; A61B 5/00

(52) U.S. Cl. CPC A61F 7/12; A61B 5/361; A61B 5/746

(57) ABSTRACT

This document describes implantable medical device systems and methods of using such devices for delivering therapy via temperature modulation. For example, this document describes medical device systems and related methods for the treatment of cardiac arrhythmias by delivering cooling to epicardial tissue. In some embodiments described herein, arrhythmias can be treated by an implantable system for painlessly terminating arrhythmias. The devices and methods provided herein permit prompt termination of atrial fibrillation almost immediately after an episode begins (to prevent persistence) and are effective irrespective of patient age and comorbidities.

### CROSS-REFERENCE TO RELATED APPLICATIONS

This application claims the benefit of U.S. Provisional Application Ser. No. 63/320,862 filed Mar. 17, 2022. The disclosure of the prior application is considered part of (and is incorporated by reference in) the disclosure of this application.

### BACKGROUND

### 1. Technical Field

This document relates to medical device systems and related methods for delivering therapy via temperature modulation. For example, this document relates to medical device systems and related methods for the treatment of cardiac arrhythmias by delivering cooling to epicardial tissue.

### 2. Background Information

Cardiac arrhythmias occur when there is a change in the rate and/or rhythm of the heartbeat due to changes in the normal sequence of cardiac electrical impulses. Abnormalities of cardiac rate and/or rhythm are associated with substantial morbidity and economic costs. Different types of cardiac arrhythmias include atrial fibrillation, bradycardia, conduction disorders, premature contractions, tachycardias, and ventricular fibrillation.

Among these conditions, atrial fibrillation is the most common arrhythmia encountered in clinical practice, affecting over 6 million Americans. Recently, cardiac arrhythmias have been reported in hospitalized coronavirus disease 2019 (COVID-19) patients, with one study reporting arrhythmias in 44% of individuals with severe illness. Studies have indicated the incidence of atrial fibrillation in the United States will increase to an estimated 12.1 million people in 2030.

Multiple prospective randomized trials have demonstrated the clinical benefit of implantable cardiac defibrillators (ICDs) in saving the lives of at-risk individuals, leading to their wide-spread adoption. A downside associated with ICD therapy, however, is the pain associated with defibrillation, whether shocks are delivered appropriately or inappropriately.

### SUMMARY

This document describes medical device systems and related methods for delivering therapy via temperature modulation. For example, this document describes medical device systems and related methods for the treatment of cardiac arrhythmias (e.g., atrial fibrillation) by delivering cooling to epicardial tissue.

In one aspect, this disclosure is directed to a medical device that includes: an implantable medical device configured to deliver cooling to adjacent tissue; an EKG detection device configured to capture EKG data from a patient; and a hand held patient control module configured to: (i) receive communications from the EKG detection device, and (ii) in response to a detection of a cardiac arrhythmia based on the EKG data, emit an alert that indicates the cardiac arrhythmia has been detected. The hand held patient control module includes a primary coil configured to transfer power to the implantable medical device by inductively coupling with a secondary coil of the implantable medical device. The implantable medical device includes a Peltier element and a heat sink comprising a phase change material.

Such a medical device may optionally include one or more of the following features. The EKG detection device may be configured to: analyze the EKG data to identify the cardiac arrhythmia; and in response to identifying the cardiac arrhythmia, transmit an alert to the hand held patient control module. The EKG detection device may be configured to transmit at least some of the EKG data to the hand held patient control module in response to identifying the cardiac arrhythmia. The EKG detection device may be configured to transmit the EKG data to the hand held patient control module. The hand held patient control module may be configured to analyze the EKG data to identify the cardiac arrhythmia. The hand held patient control module may include a rechargeable battery from which the power transferred to the implantable medical device is sourced. The hand held patient control module may be configured to transmit episodic data of the cardiac arrhythmia to a remote healthcare provider system.

In another aspect, this disclosure is directed to a method of treating a patient. The method includes: capturing EKG data from the patient using an EKG detection device; analyzing the EKG data to detect whether the EKG data is indicative of cardiac arrhythmias; emitting an alert from a hand held patient control module to indicate that a cardiac arrhythmia has been detected based on the analysis of the EKG data; inductively transferring power from a primary coil of the hand held patient control module through a skin layer of the patient and to a secondary coil of a cooling device implanted in the patient; and cooling epicardial tissue of the patient by a Peltier element within the cooling device in response to the cooling device receiving the power from the hand held patient control module.

Such a method may optionally include one or more of the following features. The analysis of the EKG data to detect whether the EKG data is indicative of cardiac arrhythmias may be performed by the EKG detection device. The method may also include sending a notification from the EKG detection device to the hand held patient control module in response to the detection that the EKG data is indicative of cardiac arrhythmias. The method may also include analyzing, by the hand held patient control module and in response to receiving the notification, whether cooling therapy should be delivered to the epicardial tissue of the patient. The method may also include transmitting at least some of the EKG data from the EKG detection device to the hand held patient control module. The analysis of the EKG data to detect whether the EKG data is indicative of cardiac arrhythmias may be performed by the hand held patient control module.

Particular embodiments of the subject matter described in this document can be implemented to realize one or more of the following advantages. First, transient therapeutic tissue cooling therapy can be delivered using the devices and methods described herein. In some embodiments, heart conditions such as arrhythmias and others can be treated using the devices and methods provided herein. In some embodiments, arrhythmias can be treated relatively painlessly. In some cases, such conditions can be treated in a minimally invasive fashion using the devices and methods provided herein. Such minimally invasive techniques can reduce recovery times, patient discomfort, and treatment costs. The implantable device described herein is advantageously powered inductively by an external device. This is advantageous because the implantable device does not require a battery, or requires a much smaller battery. Accordingly, the implantable device is smaller in size. Additionally, no batteries of the implantable device require replacement. Further, power constraints caused by an implantable device are overcome by having the ability to externally recharge the power source.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. In addition, the materials, methods and examples are illustrative only and not intended to be limiting.

The details of one or more embodiments of the invention are set forth in the accompanying drawings and the description below. Other aspects, features, and advantages of the invention will be apparent from the description, the drawings, and the claims.

### DESCRIPTION OF DRAWINGS

FIG. 1 depicts a person using an example medical device system that treats cardiac arrhythmias.

FIG. 2 is an exploded perspective view of an example implantable cooling device of the medical device system of FIG. 1.

FIG. 3 is a schematic view of an example patient control module of the medical device system of FIG. 1.

FIG. 4 is a flowchart of an example method of treating a cardiac arrhythmia using the implantable cooling device of the medical device system of FIG. 1.

Like reference numbers represent corresponding parts throughout.

### DETAILED DESCRIPTION

This document describes medical device systems and related methods for delivering therapy via temperature modulation. For example, this document describes medical device systems and related methods for the treatment of cardiac arrhythmias (e.g., atrial fibrillation) by delivering cooling to epicardial tissue.

In some embodiments, heart conditions, such as arrhythmias and others, can be treated using the devices, systems, and methods described herein. In some embodiments described herein, arrhythmias can be treated by an implantable system for painlessly terminating arrhythmias. The devices and methods provided herein permit prompt termination of atrial fibrillation almost immediately after an episode begins (to prevent persistence) and is effective irrespective of patient age and comorbidities.

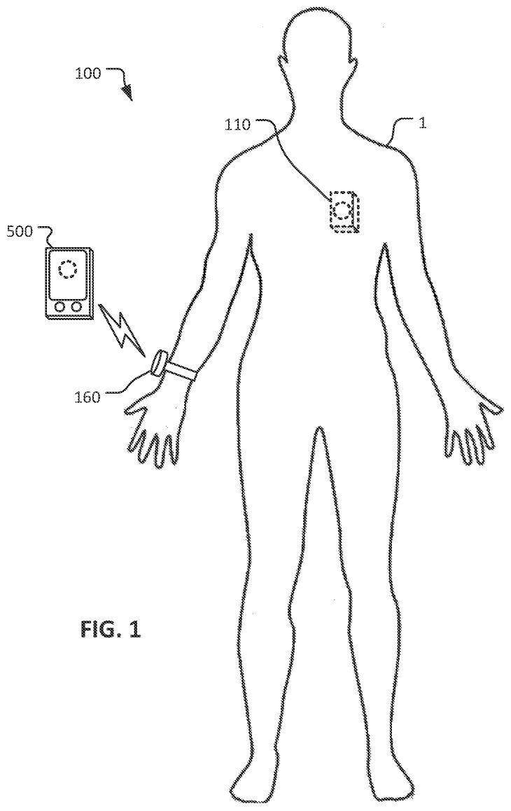

FIG. 1 depicts a patient 1 and a medical device system 100. The medical device system 100 includes an implantable medical device 110, an EKG detection device 160, and an external hand held controller device 500 (or patient control module 500). The implantable medical device 110 is implanted in the patient 1 in contact with, or proximate to, epicardial tissue. The EKG detection device 160 can monitor the electrocardiogram (“EKG”) of the patient 1. The patient control module 500 is in, or is configured to be in, wireless communication with the EKG detection device 160. The patient control module 500 is configured to be inductively coupled with the implantable medical device 110 on an as-needed basis, as described further below.

In the depicted example, the EKG detection device 160 is illustrated as a smartwatch that has an EKG detection functionality. A number of such smartwatches are currently marketed by manufacturers such as, but not limited to, FITBIT, APPLE, COROS, and SAMSUNG.

While the EKG detection device 160 is illustrated as a smartwatch, other types of devices can be used instead of a smartwatch. For example, the EKG detection devices marketed by KARDIAMOBILE can be used as the EKG detection device 160. In addition, other types of wearable EKG detection devices (e.g., bands, rings, patches, etc.) can be used as the EKG detection device 160. Further, other types of EKG detection devices that are used to monitor the EKG of the patient 1 temporarily can be used as the EKG detection device 160.

The EKG detection device 160 wirelessly communicates with the patient control module 500. In some cases, the EKG detection device 160 executes an algorithm to analyze the raw EKG data to identify arrhythmias. When the EKG detection device 160 identifies an arrhythmia (based on the analysis of the raw EKG data), the EKG detection device 160 can transmit alert information wirelessly to the patient control module 500. Alternatively, in some cases the EKG detection device 160 wirelessly communicates the raw EKG data to the patient control module 500, and the patient control module 500 can determine whether there is an arrhythmia represented in the raw EKG data. In some cases, both the EKG detection device 160 and the patient control module 500 can run algorithms to analyze the raw EKG data to identify arrhythmias.

The patient control module 500 is a multifunctional device, as described further below. For example, the patient control module 500 can communicate the status of the patient's heart rhythm to the patient 1. For example, the patient control module 500 can provide an audio, tactile, and/or visual alarm to the patient 1 when an arrhythmia is detected. Conversely, the patient control module 500 can provide a “normal” status indication to the patient 1 when the heart rhythm is normal.

The patient control module 500 can be used to activate the implantable medical device 110 to treat an arrhythmia when needed in response to the detection of an arrhythmia. In some embodiments, the patient control module 500 can determine whether cooling therapy should be delivered to the heart of the patient 1 in response to the detected arrhythmia. For example, when EKG data is indicative of an arrhythmia and the patient control module 500 determines that cooling therapy should be delivered to the heart of the patient 1, an alert can be provided to the patient 1 via the patient control module 500. In response to the alert, the patient control module 500 can be positioned by the patient 1 on, or just above, the skin surface at the location of the implantable medical device 110. In that arrangement, the patient control module 500 can become inductively coupled (transcutaneous) with the implantable medical device 110 to provide power to, and/or control of, the implantable medical device 110 when EKG data is indicative of an arrhythmia.

In order to inductively couple the patient control module 500 with the implantable medical device 110, the patient control module 500 includes a first coil (e.g., a transmitter coil) and the implantable medical device 110 includes a second coil (e.g., a receiver coil), as described further below. When the first and second coils are in sufficient proximity to each other, energy in the form of magnetic fields can be transferred between the first and second coils. In that manner, the implantable medical device 110 can receive electrical power and control signals from the patient control module 500. The power inductively received by the implantable medical device 110 from the patient control module 500 can activate a heat pump system of the implantable medical device 110. The activated heat pump system of the implantable medical device 110 can be used to cool cardiac tissue (e.g., epicardial tissue) to treat the arrhythmia. When the heart rhythm of the patient 1 has returned to normal (e.g., as detected by the EKG detection device 160), the patient 1 can simply remove the patient control module 500 from being adjacent to the location of the implantable medical device 110.

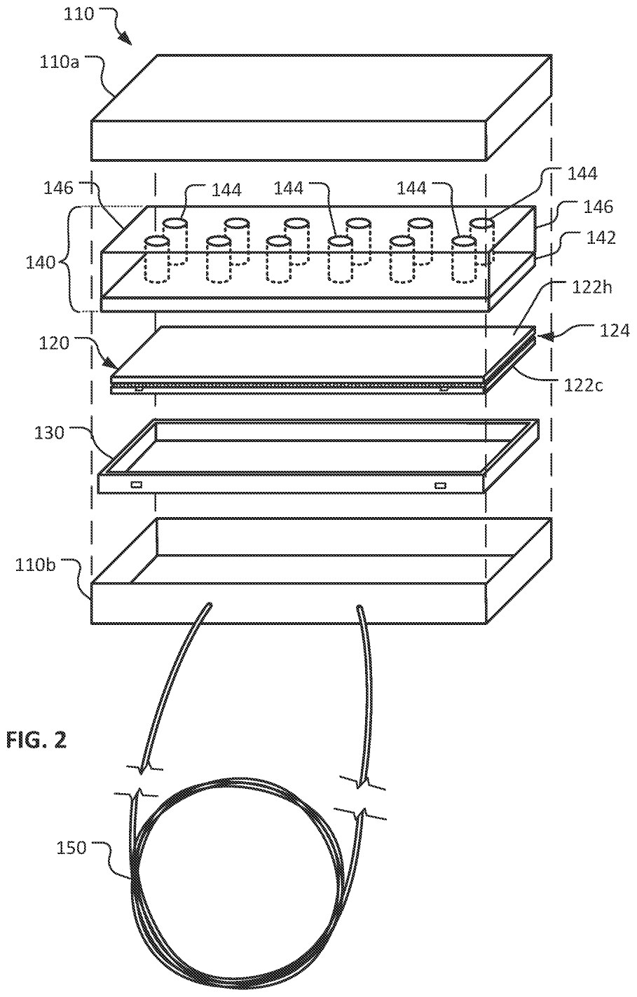

FIG. 2 is an exploded perspective view of an example embodiment of the implantable cooling device 110 that can be implanted in a human body to deliver therapeutic transient tissue cooling. The implantable medical device 110 is specifically structured to use a heat pump to deliver transient cooling to a target tissue, and to gradually dissipate the heat energy transferred from the target tissue to other body masses in a controlled and gentle manner. As described further below, a phase change material within the implantable cooling device 110 can be used to absorb the heat generated by the implantable cooling device 110 and to subsequently facilitate the gradual dissipation of the heat in a safe and effective manner.

The depicted embodiment of implantable medical device 110 (shown here in an exploded assembly view for enhanced visualization of the internal components) includes a hermetically sealable enclosure made of enclosure portions 110a and 110b, a Peltier element 120 (an example type of a heat pump), an insulative barrier 130, a heat exchange module 140 (a type of heat sink), and an electrical coil 150. The Peltier element 120, the insulative barrier 130, and the heat exchange module 140 are each hermetically sealed within enclosure. In some embodiments, the electrical coil 150 is also hermetically sealed within the enclosure. In the depicted embodiment, the electrical coil 150 extends from the enclosure (enclosure portions 110a and/or 110b) as shown.

The electrical coil 150 is used to inductively receive electrical power from the patient control module 500 to operate Peltier element 120 and/or other functionalities of the implantable cooling device 110. The electrical coil 150 can also be used to receive control signals from the patient control module 500 to control some or all of the operations of the implantable medical device 110. In some embodiments, the implantable medical device 110 can also include other components such as, but not limited to, a battery module, a wireless transceiver, a controller, one or more circuit boards, one or more electrodes (e.g., for pacing and/or for sensing EKG signals) and/or one or more physiologic sensors in, or coupled to, the enclosure portions 110a and/or 110b.

The heat pump used as part of the depicted implantable medical device 110 is the Peltier element 120. In some embodiments, other types of heat pumps can be used. In keeping with the typical construction of Peltier elements, Peltier element 120 includes a hot side 122h, and a cold side 122c. The hot side 122h and the cold side 122c are physically separated from each other and interconnected with each other by an array of alternating n-type and p-type semiconductors 124. The different types of semiconductors 124 have complementary Peltier coefficients. Semiconductors 124 are soldered between hot side 122h and cold side 122c, such that semiconductors 124 are electrically in series and thermally in parallel. As DC electric current flows through Peltier element 120 (as sourced from the electrical coil 150 that is electrically connected to Peltier element 120), heat from cold side 122c is transferred to hot side 122h, so that cold side 122c gets cooler while hot side 122h gets hotter.

In the depicted embodiment, the peripheral edges of the Peltier element 120 are insulated from the enclosure portions 110a and/or 110b by the insulative barrier 130. The insulative barrier 130 can be just an electrical insulator, or just a thermal insulator, or both an electrical and a thermal insulator. The insulative barrier 130 can be made of any suitable insulative material such as, but not confined to, Teflon®, phenolic cast resins, nylon, glass and the like. In some embodiments, the insulative barrier 130 also acts to hold the Peltier element 120 in place so that the cold side 122c stays in direct contact with the enclosure portion 110b while also leaving a space between the edges to allow for welding shut of the enclosure portions 110a and 110b. In some embodiments, the cold side 122c is thermally coupled with the enclosure portion 110b via a low thermal resistance material/structure positioned between the cold side 122c and the inner wall surface of the enclosure portion 110b.

The Peltier element 120 is hermetically sealed within the enclosure portions 110a and 110b to protect the Peltier element 120 from body fluid ingress. Any biologically inert, highly heat conductive metal can be used to construct the enclosure such that the Peltier element 120 is isolated from body fluids. Such a biologically inert, highly conductive metal for the enclosure portions 110a and 110b can include, but is not limited to, titanium, titanium alloys, stainless steel, stainless steel alloys, 316 stainless steel, and the like, and combinations thereof. In some embodiments, the enclosure portions 110a and 110b are welded closed in a hermetically sealed fashion. The enclosure is also equipped with a means of allowing electrical wires of the electrical coil 150 to exit the enclosure through glass feedthroughs, for example.

While the depicted embodiment of the implantable medical device 110 includes a rectangular enclosure, it should be understood that other form factors are also envisioned. For example, in some embodiments one or both of the enclosure portions 110a and 110b have a contoured outer surface rather than a planar outer surface. In some such embodiments, the enclosure portions 110a and/or 110b can be specifically contoured to interface with a particular patient's anatomy.

As needed, the electrical coil 150 can be temporarily energized to provide a source of DC energy to power the Peltier element 120. In some embodiments, a battery module (not shown) is also included attached to, or within, in the implantable medical device 110. Such a battery module can also include a controller and/or one or more physiologic sensors in, or coupled to, the same enclosure as the battery module. In particular embodiments, the electrical coil 150 is implanted/positioned within the patient 1 just under the patient's skin tissue layer. The electrical coil 150 is configured to inductively receive DC energy from the coupleable primary coil of the patient control module 500, either transiently or on an on-going basis.

During operation of the implantable medical device 110, some or all of the outer surface of the enclosure portion 110b will become cooled by virtue of its thermal contact with the cold side 122c of the Peltier element 120. In some embodiments, the actual portion of the enclosure portion 110b in contact or thermal communication with the cold side 122c is thermally insulated from other portions of the enclosure portion 110b (and the enclosure portion 110a). The cooled portion of the enclosure portion 110b can be placed into contact with the target tissue (e.g., the epicardium, in the example of cardiac therapy).

As described herein, while the Peltier element 120 cools on cold side 122c (and enclosure portion 110b), the other side (the hot side 122h) of the Peltier element 120 heats up. Some dissipating or managing of the heat on the hot side 122h is needed to prevent injury to the tissues in contact with the enclosure portion 110a (the hot side of implantable medical device 110). The heat exchange module 140 is specifically designed for this purpose. In some embodiments, the temperature of the cold side 122c ranges from about 1 degree Celsius to about 37 degrees Celsius during operation. In some embodiments, the temperature of the hot side 122h ranges from about 37 degrees Celsius to about 50 degrees Celsius during operation. In some embodiments, the therapeutic cold temperature ranges from about 5 degree Celsius to about 15 degrees Celsius.

In some embodiments, the therapeutic period of time during which cooling is delivered ranges from about 5 seconds to about 180 seconds. In some embodiments, the cold enclosure portion 110b is in contact with an epicardial tissue and the warm enclosure portion 110a is in contact with a pericardial tissue when the implantable medical device 110 is implanted.

The heat sink of implantable medical device 110 (i.e., the heat exchange module 140) is constructed so that heat is initially absorbed and then subsequently released to body tissues (or blood, other body fluids, etc.) in contact with the enclosure portion 110a gradually, such that the temperature of the enclosure portion 110a (the hot side of the implantable medical device 110) is not significantly above body temperature. In some embodiments, the implantable medical device 110 is designed to keep the outside of the implantable medical device 110 exposed to body tissue below 50 degrees centigrade, and more ideally closer to 40 degrees centigrade. Accordingly, implantable medical device 110 is configured to permit rapid cooling without injury to the patient's tissues in contact with the hot side (e.g., the enclosure portion 110a), and without the need for external cooling fins or other heat dissipation mechanisms.

Again, the functionality of the medical device 110 includes the utilization of the heat exchange module 140 that interfaces with the hot side 122h of the Peltier device 120 through direct contact or via a thermally conductive interface material/structure. The heat exchange module 140 also interfaces with the enclosure portion 110a (the hot side of implantable medical device 110). In some embodiments, one or more portions of the heat exchange module 140 is in direct contact with one or more inner wall portions of the enclosure (e.g., enclosure portion 110a). In some embodiments, an insulative barrier is positioned between one or more portions of the heat exchange module 140 and one or more inner wall portions of the enclosure.

In the depicted embodiment, the thermally conductive interface structure of the heat exchange module 140 includes a plate 142 and a plurality of columns 144 extending from the plate 142. In some embodiments, the plate 142 and the columns 144 may be constructed of very highly thermally conductive materials (e.g., having a thermal conductivity of k=200 to 300 W/m-deg C or higher), such as Annealed Pyrolytic Graphite (APG) or other highly conductive material alloy structures to accelerate heat transfer from the hot side 122h of the Peltier device 120 into the heat exchange module 140. The heat exchange module 140 (being constructed of APG or other highly conductive material) is designed to rapidly conduct the heat from the hot side 122h of the Peltier device 120, and to evenly distribute the heat through the PCM 146 (since the PCM 146 itself typically will have a very low thermal conductivity).

While the columns 144, as depicted, have circular cross-sectional shapes, in some embodiments the columns 144 have other cross-sectional shapes such as, but not limited to, triangular, rectangular, polygonal, ovular, star-shaped, and the like. While the depicted embodiment includes twelve columns 144, in some embodiments one, two, three, four, five, six, seven, eight, nine, ten, eleven, thirteen, fourteen, fifteen, sixteen, seventeen, eighteen, nineteen, twenty, or more than twenty columns 144 are included.

The heat exchange module 140 also includes a phase change material (PCM) 146. The PCM 146 is in contact with the plate 142 and in contact with the plurality of columns 144. In the depicted embodiment, the plurality of columns 144 extend into the PCM 146.

In some embodiments, the PCM 146 is made of a material with a melting temperature in the range of between human body core temperature (37° C.) and 50° C. PCMs that are applicable due to their high specific heat, high heat of fusion and melting temperature in the range of about 37° C. and 50° C. include, but are not limited to, paraffins. In some embodiments, the PCM 146 has a very low thermal conductivity (e.g., k=0.2 W/m-deg C). Some desirable characteristics of the PCM 146 are low volume change with phase change, no toxicity, no corrosivity, and compatibility with the material of enclosure.

A particular desirable property of the PCM 146 is that it absorbs heat while exhibiting a minimal rise in temperature. In some embodiments, the PCM 146 can absorb heat without exhibiting a sensible temperature rise as it changes phase from solid to liquid, or liquid to vapor. In particular embodiments, the PCM 146 is capable of absorbing more than twice the heat energy of non-PCMs that do not undergo phase change. Two additional properties of the PCM 146 are high specific heat (Cp) and high heat of fusion (h). Specific heat is the amount of heat a material can absorb per unit mass. Heat of fusion is the amount of heat a material can absorb per unit mass without a sensible temperature increase (at constant temperature) while undergoing a change of phase, for example, transitioning from a solid to a liquid, or a liquid or solid to a gas (vapor).

The heat sink and heat exchange structures/properties of the implantable medical device 110 involves three main components: (i) the metal enclosure 110a-b, (ii) the PCM 146, and (iii) the thermally conductive interface of the heat exchange module 140 (which includes the plate 142 and the plurality of columns 144 extending from the plate 142) that is interfaced with the hot side 122h of the Peltier device 120. In some embodiments, the columns 144 may consist of rods or fins designed to efficiently and rapidly transport the heat from the plate 142 and the hot side 122h into the PCM 146. In some embodiments, the columns 144 may extend into the PCM 146 between approximately the midpoint of the heat exchange module 140 to the very top of the thickness of the PCM 146 (or just short thereof).

In some embodiments, the enclosure 110a-b and/or the electrical coil 150 comprises one or more fixation elements configured to secure the enclosure 110a-b and/or the electrical coil 150 to adjacent tissue. The one or more fixation elements can be barbs, helical elements, roughened surface portions, tines, hooks, and the like, and combinations thereof.

Optionally, the implantable medical device 110 includes one or more temperature sensors located within, or remote from, the enclosure 110a-b. For example, in some embodiments the implantable medical device 110 can include a first temperature sensor and a second temperature sensor that are configured to measure the temperature of the cold enclosure portion 110b and the warm enclosure portion 110a. As such, the first and second temperature sensors monitor the temperatures of the implantable medical device 110. Alternatively, the first and second temperature sensors may monitor the temperature of the tissue surrounding the implantable medical device 110 once implanted. The first and second temperature sensors can provide feedback to a processor and/or controller of the implantable medical device 110 or the patient control module 500 (e.g., information regarding cooling efficacy, unwanted heating, and the like).

In some embodiments, the implantable medical device 110 optionally further includes one or more pacing electrodes attached to, or on one or more leads extending from, the enclosure 110a-b. In some embodiments, the implantable medical device 110 further includes one or more EKG sensing electrodes disposed on the enclosure 110a-b and/or on one or more leads extending from the enclosure 110a-b. In some embodiments, the implantable medical device 110 is, or comprises, one or more of an implantable cardioverter defibrillator (ICD), a pacemaker, or a heart monitor.

The implantable medical device 110 and/or the patient control module 500 can include several programmable parameters such as the cooling temperature set point and the duration of the cooling therapy. Programmable parameters of the cooling therapy may include a cooling temperature set point (e.g., about 5 degrees Celsius to about 15 degrees Celsius), a duration (e.g., on time) (e.g., about 5 seconds to about 180 seconds), a therapy target cooling temperature range (e.g., a minimum and a maximum therapeutic target temperature) (e.g., about 15 degrees Celsius to about 5 degrees Celsius), a maximum (e.g., a maximum threshold) cool temperature (corresponding to the cold surface of the housing portion 110b) (e.g., about 5 degrees Celsius to about 0 degrees Celsius), a maximum (e.g., a maximum threshold) warm temperature (corresponding to the hot surface of the housing portion 110a) (e.g., about 45 degrees Celsius to about 55 degrees Celsius), a maximum (e.g., a maximum threshold) energy or power delivery (e.g., voltage). A safety termination of cooling therapy (e.g., the implantable medical device 110 is turned off) may result if one of the following conditions is met: the maximum cold threshold temperature is exceeded (corresponding to the cold surface of the housing portion 110b) (e.g., about 5 degrees Celsius to about 0 degrees Celsius), the maximum hot threshold temperature is exceeded (corresponding to the hot surface of the housing portion 110a) (e.g., about 45 degrees Celsius to about 55 degrees Celsius), the maximum threshold energy or power delivery is exceeded (e.g., about 200 J to about 500 J). Alternatively, the therapy may comprise delivering a proscribed voltage of a proscribed pulse width, for a specific period of time, and the therapy may be delivered for a specific period of time while monitoring for excessive cooling or heating, and the therapy turned off after that period of time.

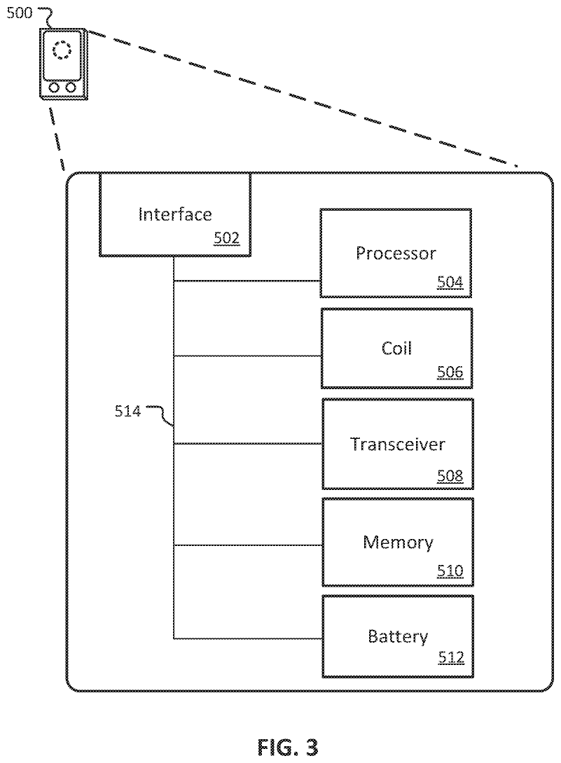

FIG. 3 schematically depicts the patient control module 500 that receives biometric (e.g., EKG) data of the patient 1 (FIG. 1) and transmits signals and/or energy through transcutaneous inductive coupling with the implantable medical device 110. For example, the patient control module 500 is inductively coupleable to the implantable medical device 110 illustrated in FIGS. 1 and 2, and powers the implantable medical device 110 wirelessly through inductive power transmissions.

The patient control module 500 includes components such as, but not limited to, an interface 502, a processor 504, a coil 506, a transceiver 508, a memory 510, a battery 512, and a bus 514. The described illustration is only one possible implementation of the described subject matter and is not intended to limit the disclosure to the single described implementation of the patient control module 500. Those of ordinary skill in the art will appreciate the fact that the described components can be connected, combined, or used in alternative ways, consistent with this disclosure.

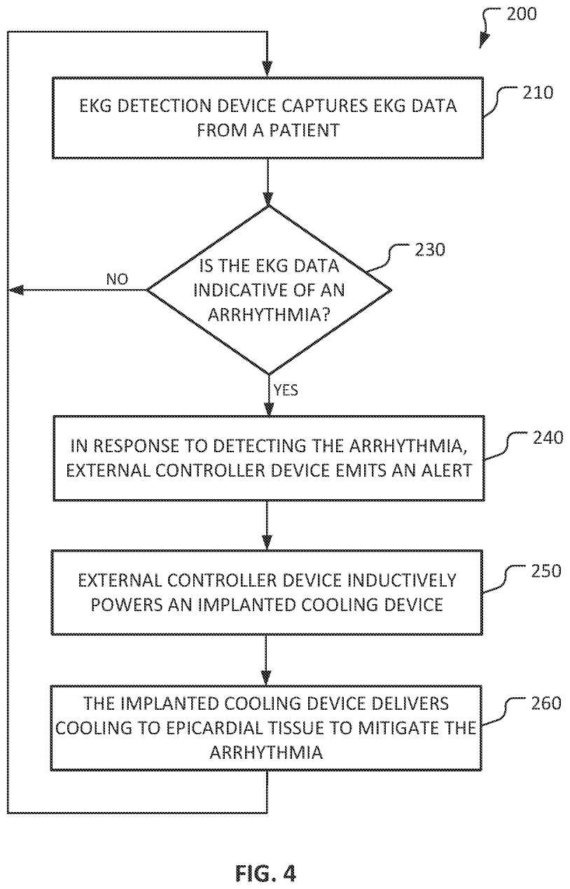

The patient control module 500 includes a computing system configured to perform one or more algorithms to be executed by the implantable medical device 110. For example, the patient control module 500 includes the algorithm to activate the implantable medical device 110 to perform the method 400 described in FIG. 4. In some embodiments, the algorithm can be implemented in an executable computing code or an application program. The algorithm can be stored in the memory 510.

In some embodiments, the patient control module 500 may be in wireless communication with an input device, such as a touchpad, a wearable device, or any other type of device that detects biometric data (such as the EKG detection device 160), to receive the biometric data of the patient 1. The input device (such as the EKG detection device 160) includes one or more sensors, e.g., the sensing electrodes described in FIG. 1, to detect one or more biometric signals from the patient 1.

In some embodiments, the patient control module 500 can serve as a client device, and one or more components of the patient control module 500 can be configured to operate within a cloud-computing based environment to sync the biometric data of the patient 1 uploaded from the patient control module 500 via a network. That is, the patient control module 500 can be an electronic device operable to receive, transmit, process, store, or manage data, and is communicably coupled with a server managed by a third party.

A user (such as the patient 1) may operate the patient control module 500 via the interface 502, and each of the components of the patient control module 500, such as the interface 502, the processor 504, the coil 506, the transceiver 508, the memory 510, the battery 512, can communicate using the bus 512. For example, the user may review the biometric data collected by the input device or the status of the implantable medical device 110 received by the transceiver 508 through the interface 502. When the patient 1, or the patient control module 500, observes an abnormal situation (e.g., atrial fibrillation), the patient 1 may determine to execute a specific procedure, e.g., the cooling therapy using the implantable medical device 110.

In some embodiments, the coil 506 may be a transmitting coil that transfers power provided by the battery 512 to power or charge the implantable medical device 110 wirelessly. Upon the patient control module 500 approaching the implantable medical device 110 within a charging range, the interface 502 of the patient control module 500 may display a status of the implantable medical device 110, such as a power transmission status of the implantable medical device 110, a status of the cooling therapy, user's biometric data, and/or data from the implantable medical device 110. In some embodiments, the patient control module 500 can be programmed to activate the implantable medical device 110, such as beginning the cooling therapy, upon detecting that the patient control module 500 is within the range for induction power transmission with the implantable medical device 110.

The processor 504 executes instructions and processes data to perform the operations of the patient control module 500, and the operations of the medical device system 100 as a whole. In some embodiments, the processor 504 can include a data processing apparatus to process the biometric data (e.g., EKG data) of the patient 1 to provide a determination of whether atrial fibrillation is occurring.

The memory 510 of the patient control module 500 can record biometric data of the patient 1 collected from the input device (e.g., the EKG detection device 160) and/or the implantable medical device 110. For example, the patient control module 500, or a third party, can retrieve historical biometric data of the patient 1 from the memory 510 as a reference or an input in various circumstances, such as processing the primary determination, predicting a prognosis, and training a machine-learning model. While the memory 510 is illustrated as an integral component of the patient control module 500, in an alternative embodiment, the memory 510 can be external to the patient control module 500.

FIG. 4 is a flowchart of a method 200 for treating a cardiac arrhythmia in a patient in need thereof, in accordance with some embodiments provided herein. In some embodiments, the method 200 can be performed by, or using, the implantable medical device system 100 described herein.

In step 210, an EKG detection device captures EKG data from a patient. As described above, in some embodiments the EKG detection device is a wearable device. However, any type of EKG detection device can be used for performing the method 200.

In step 230, the EKG data captured from the patient is analyzed to determine whether the EKG data is indicative of a cardiac arrhythmia. The EKG detection device and/or an external controller device can run one or more algorithms to analyze the captured EKG data. The algorithms can be designed to identify one or more types of an irregular pattern of the received EKG data. That is, the one or more algorithms can be used to determine whether the EKG data captured by the EKG detection device indicates that the patient is experiencing one or more types of cardiac arrhythmia.

In some embodiments, the analysis of the EKG data is performed by the EKG detection device that also captured the EKG data from the patient. In such a case, in response to the detection of an arrhythmia by the EKG detection device, an alarm/alert can be transmitted from the EKG detection device to an external handheld patient control device (such as the patient control module 500 described above). In some embodiments, at least some of the EKG data can also be transmitted from the EKG detection device to the patient control device. The transmission(s) between the EKG detection device and the patient control device can be made wirelessly, or by wire. Wireless transmissions can be made using any suitable technology such as, but not limited to, Bluetooth, Wi-Fi, RFID, NFC, RF, IR, Zigbee, and the like.

In certain embodiments, in response to the receipt of the alarm/alert and/or the receipt of the EKG data, the patient control device can analyze whether or not cooling therapy should be delivered to the heart of the patient in response to the detection of the arrhythmia. Such analysis can be based on factors such as, but not limited to, the type of alarm/alert, the EKG data, one or more threshold parameters (which can be customized for the particular patient), patient history, and the like.

Optionally, in some embodiments the analysis of the EKG data is performed by the patient control device (such as the patient control module 500 described above) that receives the EKG data from the EKG detection device. In some embodiments, the analysis of the EKG data is performed by both the EKG detection device and the patient control device.

In step 240, in response to a detection by the external patient control device that the patient is experiencing a cardiac arrhythmia, the external patient control device can emit an alert or an alarm to indicate that a cardiac arrhythmia is occurring. The alert/alarm can be audible, tactile, visual, and/or combinations thereof. In some embodiments, an alert/alarm can be sent to one or more remote receivers via Wi-Fi or a cell phone transmission. The remote receivers can be that of a caretaker, physician, clinic, healthcare provider, and the like.

In some embodiments, step 240 also includes (in addition to the detection of the arrhythmia) an analysis by the patient control device to determine whether cooling therapy should be delivered in response to the detection of the arrhythmia. Such analysis can be based on factors such as, but not limited to, the type of alarm/alert, the EKG data, one or more threshold parameters (which can be customized for the particular patient), patient history, and the like.

In some embodiments, the alert emitted by the external patient control device can include information regarding the detection of the arrhythmia (e.g., displayed on a display device of the external patient control device). The display device (or other aspects of the user interface of the external patient control device) can also facilitate user input to the external patient control device. For example, in some embodiments the user can enter a selection to indicate whether or not the user desires the cooling therapy to be delivered.

In step 250, the external controller device can be used to inductively power an implanted cooling device of the patient experiencing the cardiac arrhythmia. The implanted cooling device can be any of the embodiments of the implanted cooling device 110 described herein. For example, as explained in the context of FIG. 1 and elsewhere herein, the external controller device can be placed in close contact (e.g., adjacent to a skin surface) to the location of the implanted cooling device of the patient. In doing so, the primary coil of the external controller device can transmit electrical energy to the secondary coil of the implanted cooling device in order to power/energize the implanted cooling device in accordance with a treatment protocol. In some embodiments, control signals in accordance with the treatment protocol can also be transmitted wirelessly from the external controller device to the implanted cooling device via the coils. Moreover, in some embodiments data can be transferred from the implanted cooling device to the external controller device via the coils.

In step 260, the implanted cooling device (being energized by the power received from the external controller device) can deliver cooling to epicardial tissue of the patient to mitigate the occurrence of the cardiac arrhythmia. For example, implanted cooling device 110 described herein includes a Peltier element that, when energized, transfers heat from a cool side to a warm side of the implanted cooling device. The cool side can be in thermal contact with the epicardial tissue of the heart of the patient. Such cooling of the epicardial tissue can serve to mitigate the occurrence of the cardiac arrhythmia. Meanwhile, the EKG detection device can continue to capture EKG data from the patient, and to transfer the EKG data to the external controller device. When the captured EKG data is no longer indicative of a cardiac arrhythmia, the external controller device can stop delivering power to the implanted cooling device so that the implanted cooling device stops cooling the epicardial tissue of the patient.

After use of the external controller device, the battery of the external controller device can be recharged. Such recharging can be performed inductively or by plugging in a wire to recharge the battery.

In some embodiments, after the use of the external controller device, episodic data stored by the external controller device pertaining to the usage of the external controller device and/or the implanted cooling device to deliver cooling to treat an arrhythmia can be transmitted from the external controller device to a healthcare provider. The transmission can be performed via WiFi, Bluetooth, a cellular signal, and any other suitable mode of communication.

### Additional Optional Features

In some embodiments provided herein, implantable thermal therapy device 110 includes temperature monitoring devices (e.g., integrated thermocouples, thermistors, or other types of temperature monitoring devices) for temperature registration and feedback.

In some embodiments, implantable thermal therapy device 110 can be affixed to a non-biodegradable fabric to permit surgical suturing of thermal therapy device 110 to target tissues or to increase friction with adjacent tissues.

In some embodiments, the cooling module is patient activated. In some embodiments, the cooling module is activated automatically if one or more conditions is/are detected (e.g., by sensors in communication with the implanted thermal therapy device) that indicate that providing tissue cooling will be beneficial to the patient.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment.

Conversely, various features that are described in the context of a single embodiment can also be implemented in multiple embodiments separately or in any suitable sub-combination. Moreover, although features may be described herein as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results.

What is claimed is:

1. A medical device system comprising:
an implantable medical device configured to deliver cooling to adjacent tissue, wherein the implantable medical device comprises a Peltier element and a heat sink comprising a phase change material;
an EKG detection device configured to capture EKG data from a patient; and
a hand held patient control module configured to: (i) receive communications from the EKG detection device, and (ii) in response to a detection of a cardiac arrhythmia based on the EKG data, emit an alert that indicates the cardiac arrhythmia has been detected,
wherein the hand held patient control module includes a primary coil configured to transfer power to the implantable medical device by inductively coupling with a secondary coil of the implantable medical device.

2. The medical device system of claim 1, wherein the EKG detection device is configured to:
analyze the EKG data to identify the cardiac arrhythmia; and
in response to identifying the cardiac arrhythmia, transmit an alert to the hand held patient control module.

3. The medical device system of claim 2, wherein the EKG detection device is further configured to transmit at least some of the EKG data to the hand held patient control module in response to identifying the cardiac arrhythmia.

4. The medical device system of claim 2, wherein the EKG detection device is configured to transmit the EKG data to the hand held patient control module, and wherein the hand held patient control module is configured to analyze the EKG data to identify the cardiac arrhythmia.

5. The medical device system of claim 1, wherein the hand held patient control module includes a rechargeable battery from which the power transferred to the implantable medical device is sourced.

6. The medical device system of claim 1, wherein the hand held patient control module is configured to transmit episodic data of the cardiac arrhythmia to a remote healthcare provider system.

7. A method of treating a patient, the method comprising:
capturing EKG data from the patient using an EKG detection device;
analyzing the EKG data to detect whether the EKG data is indicative of cardiac arrhythmias;
emitting an alert from a hand held patient control module to indicate that a cardiac arrhythmia has been detected based on the analysis of the EKG data;
inductively transferring power from a primary coil of the hand held patient control module through a skin layer of the patient and to a secondary coil of a cooling device implanted in the patient; and
cooling epicardial tissue of the patient by a Peltier element within the cooling device in response to the cooling device receiving the power from the hand held patient control module.

8. The method of claim 7, wherein the analysis of the EKG data to detect whether the EKG data is indicative of cardiac arrhythmias is performed by the EKG detection device.

9. The method of claim 8, further comprising sending a notification from the EKG detection device to the hand held patient control module in response to the detection that the EKG data is indicative of cardiac arrhythmias.

10. The method of claim 8, further comprising analyzing, by the hand held patient control module and in response to receiving the notification, whether cooling therapy should be delivered to the epicardial tissue of the patient.

11. The method of claim 8, further comprising transmitting at least some of the EKG data from the EKG detection device to the hand held patient control module.

12. The method of claim 7, wherein the analysis of the EKG data to detect whether the EKG data is indicative of cardiac arrhythmias is performed by the hand held patient control module.
